(19) United States
(12) Patent Application Publication
(10) Pub. No.: US 2023/0240478 A1
(43) Pub. Date: Aug. 3, 2023

(54) VIRTUAL PANEL FOR CEILINGS AND/OR WALLS WITH WATER AND/OR AIR

(71) Applicant: Irina Bontoi, Badalona (Barcelona) (ES)

(72) Inventor: Irina Bontoi, Badalona (Barcelona) (ES)

(21) Appl. No.: 18/010,293

(22) PCT Filed: Jun. 8, 2021
(86) PCT No.: PCT/ES2021/070421

§ 371 Date: Dec. 14, 2022

(30) Foreign Application Priority Data
Jun. 15, 2020 (ES) U202031261
Oct. 28, 2020 (ES) U202032340

(51) Int. Cl. A47K 3/28; G09F 19/22; G09F 13/22; G09F 27/00; A61H 33/00

(52) U.S. Cl. CPC A47K 3/281; G09F 19/22; G09F 13/22; G09F 27/005; A61H 33/6063; G09F 2013/222

(57) ABSTRACT

A virtual panel for ceilings and/or walls with water and/or air is provided which can be installed as an accessory in an existing room (2) or during the construction of said room and which comprises an electrical or electronic device (10, 12, 16) which, when activated, displays a moving image of a virtual representation of the sky or other natural or non-natural scenery. The panel includes at least one outlet nozzle (6) for fluid, which may be water and/or air, depending on the type of room (2). The room (2) may include a receptacle (25) for housing containers (28) with essences, which incorporates a suction device (27) for emitting odours through the air nozzles (6). Concealing glass or smart foil may be provided on the screen (10) or ceiling (12) with lights (13), which becomes opaque or translucent when an electric current is applied thereto.

### OBJECT OF THE INVENTION

As stated in the title of the present specification, the invention relates to a virtual panel for ceilings and/or walls of rooms with water and/or air, contributing advantages and features to the function intended, which are described in detail further on.

The object of the present invention relates to a panel which can be installed in a ceiling and/or in one or more walls of a room, with water and/or air.

This invention is of particular interest, without ruling out other possibilities, for a room comprising a shower or bathtub, as well as for a lift car.

This invention can also be of interest, for example, for a cabin used in massage, aesthetic treatments, and dentistry.

In conclusion, the invention may be of interest for any room in which a user spends a certain amount of time performing a specific activity, such as going up in a lift or taking a bath or shower.

The virtual panel for ceilings and/or walls with water and/or air stands out due to the fact that it is installed as an accessory device which covers at least the upper portion or the ceiling of the room and/or simultaneously one or more of its walls, further comprising at least one outlet for a fluid, which can be water and/or air, for the purpose of providing the image of a virtual representation of a scenery, such as the sky or other preferably natural sceneries, such that the user, when using the room, has the feeling of being outdoors, immersed in said scenery, instead of being inside a small closed enclosure.

### FIELD OF APPLICATION OF THE INVENTION

The field of application of the present invention is comprised within the sector of construction, particularly encompassing the sector of the industry dedicated to the manufacturing and installation of ceilings and/or walls with water and/or air of different types, and specifically at least relating to bathrooms and lifts, encompassing at the same time the sector of imaging apparatus, devices, and equipment.

### BACKGROUND OF THE INVENTION

As a reference to the current state of the art, it should be noted that at least the applicant is unaware of the existence of any other virtual panel for ceilings and/or walls with water and/or air or any other invention of similar application, which presents technical and structural features that are the same or similar to those presented by that which is claimed herein.

### SUMMARY OF THE INVENTION

The virtual panel for ceilings and/or walls with water and/or air proposed by the invention and as indicated above is an electric or electronic accessory device which can be installed at least in the upper portion or the ceiling of a room, and optionally in one of the walls of the room, for the purpose of providing the moving image of a virtual representation of a scenery, such as the sky or another natural scenery, whether by day or by night, such that, along with the emission of a fluid in the form of water and/or air through one or more outlets provided to that end, the user will have the feeling of being outdoors when he or she uses the room and activates the panel.

As mentioned, the room for which the virtual panel of the invention is intended is preferably a room comprising a shower or a bathtub, in which case it may have a water and/or air outlet, or a lift car, in which case, logically, the outlet for a fluid will only be one for air, emulating a pleasant breeze programmed within a time interval or a strong breeze in case of emergency, for example.

Nevertheless, the application of the invention in other types of room is not ruled out, such as a cabin used in massage, a cabin used in aesthetic treatments, a cabin used in dentistry, or any other in which the user spends a certain amount of time performing a specific activity.

In any case, the virtual panel for ceilings and/or walls with water and/or air is an additional accessory which can be installed in an existing room or during the construction of said room.

Moreover, the virtual panel is preferably designed in three options,

1. a first option in which the panel covers only the ceiling of the room, with the images it shows preferably being the images of a moving sky, be it by day and/or by night,

2. a second option in which, in addition to the ceiling of the room, the panel covers a portion or the entirety of one of the walls of the room, with the images it shows in such case being optionally the images of a day or night sky, or the images of other sceneries, for example, a waterfall if the room is a shower or a bathtub, and

3. a third option in which, in addition to the ceiling, the virtual panel covers two or more walls of the room, with the images it shows being the images of a moving sky or other moving sceneries.

In any case, the images shown on the panel are always moving images, for example, moving clouds and sun, in order to emulate the day sky, or moving stars and moon, in order to emulate the night sky, and to thereby make the user feel as if he or she is in the natural scenery depicted by the images.

It is important to point out that the virtual images shown on the panel can be images with moving lights, or pre-recorded video images, or projected 3D images, or holograms, or a platform complemented with a network of moving mini lights forming 3D images.

To that end, the virtual panel of the invention comprises at least one image display screen which shows the moving image when activated by means of a digital control unit preferably integrated in the wall of the room and in the form of a window.

Additionally, the panel also incorporates a pleasant sound that fits the image displayed on the screen, for example, the sound of the wind, the sound of the sea, the sound of birds, the sound of crickets, etc., so as to increase the user's feeling of being immersed in the scenery depicted by said image.

Moreover, it should be pointed out that, depending on the dimensions of the room, the panel can be made up of a single screen to only cover the ceiling or can be made up of a plurality of screens placed next to one another covering the ceiling and, where appropriate, the wall or walls of the room, collectively showing the virtual image in question as if they are a single screen.

Another embodiment of the panel is a platform in which there is a plurality of LED lights connected to one another which collaborate with one another in a continuous and well-balanced manner, making a complex link forming the moving image pre-recorded in the software as some lights turn off and others gradually turn on.

Preferably, the digital control unit regulating the actuation of the panel and/or optionally of the water flow, which is incorporated in the wall of the room, is integrated in a small touchscreen which is in turn in the form of a window arranged in the wall where the same moving image of the virtual panel in the ceiling is displayed in the background.

In a preferred embodiment, when the customer, according to his or her tastes, wants, or needs, can select and change the moving images of the panel by means of a wireless key which will upload the new moving images into the digital control unit. Optionally, the customer will receive said key or portable device at home after purchasing it online. The user will only have to bring the key close to the digital control unit for the new image to be downloaded into the software and for it to be displayed starting from said moment on the panel of the ceiling and/or walls.

Optionally, the digital control unit comprises manual activation such that the user may or may not start the operation of the panel as he or she wishes. This option is preferably envisaged for the installation of the virtual panel in a room comprising a shower or bathtub and is in the form of a window.

In another alternative or complementary option, the panel is automatically activated as the user enters the room as a result of the existence of a movement or presence sensor. This option is preferably envisaged for when the panel is installed in a lift.

Furthermore, preferably, the digital control unit is connected to the conventional lighting of the room in which the panel is arranged, for example, in a room comprising a shower or bathtub, such that, when activated, the intensity of light is automatically reduced by about 50% or more, creating a more authentic ambient.

### Preferred Embodiment in Which the Room is a Lift Car

Likewise, also in an optional manner, and particularly when the room is a lift car and the panel completely or partially covers a wall thereof, a portion of the panel is made up of a touchscreen such that, when double-clicking said screen, it stops displaying a portion of the virtual image and transforms into a mirror, and by double-clicking it again, it recovers the full display of the moving image, such that users of the lift can choose between looking at themselves in the mirror or allowing the virtual image to completely surround them while they go up or down. Logically, said screen which can be transformed into a mirror will be suitably located at the eye level of an adult, where it can have a larger or smaller dimension.

In turn, the exit of the fluid, in this case preferably air, occurs through a series of openings provided by way of nozzles, for example, in parts arranged for that purpose at the edges of the frame of the screen or screens comprised in the panel, or facing same, with the corresponding fluid conduit connection.

The air exiting through the nozzles originates from a mini air conditioning, preferably coupled above the lift car, injecting air into the room.

The start-up and activation or operation of the panel of the lift will preferably be carried out by means of the aid of a manual control. This manual control is preferably independent of the control unit of the lift.

Furthermore, in an embodiment option, the invention contemplates the introduction of a system for emitting odours through the air outlet nozzles, for which there is envisaged in the room the existence of a receptacle into which a container with an essence can be introduced, for example, vanilla, menthol, etc., and with a device which suctions the essence and discharges it into the room through the air nozzles.

In a preferred embodiment, the inclusion of an emergency button is envisaged which, when pressed in case of need, in addition to activating the alarm warning in a remote emergency centre and sending the actual location of the user, also activates, at the same time, the device for emitting odours along with the increase discharge of fresh air through the nozzles of the panel, as well as the playback of a video in which a person performs meditation and breathing exercises for several minutes in an attempt to calm the user in emergency and/or jam-up situations.

### Preferred Embodiment in Which the Room Comprises a Shower or Bathtub

Likewise, also in an optional manner, and particularly when the room comprises a shower or bathtub, the panel comprises an upper screen which is incorporated built into the ceiling above a platform with water outlet nozzles which is in turn manufactured in a transparent material, such that it allows viewing the moving image shown on the screen therethrough. Optionally, said platform is a drop-down platform to facilitate cleaning the screen.

Optionally, the panel of the invention comprises a false ceiling in which there is integrated a plurality of small lights, for example, LED lights, which are connected to one another and to a motor defining a rotational movement thereof, for example, about a support disc, such that it emulates the movement of the stars, moon, sun, clouds in the sky or cosmos.

In this case, like in the preceding case, the false ceiling with lights preferably covers the entire upper portion of the shower or bathtub and is coupled built into the ceiling on the platform with water outlet nozzles, which is in turn preferably formed by a part manufactured in a transparent material, such as glass, methacrylate, or polycarbonate, such that it allows viewing the image shown on said false ceiling therethrough, which platform with water outlet nozzles is optionally a drop-down platform to allow cleaning.

Preferably, in any of the two mentioned variants, when the room is a shower or bathtub, on the screen or false ceiling with lights, the existence of a concealing glass or smart foil is envisaged, of the type which becomes opaque or translucent when an electric current is applied thereto, such that, when the screen or the false ceiling with lights is inactive, said glass becomes opaque and conceals the screen or the false ceiling being integrated in the ceiling.

In another embodiment variant also applicable in a room comprising a shower or bathtub, the panel of the invention comprises a set of side projectors attached to the panel which, when turning on the panel, move and are oriented with a suitable inclination, for example of 45°, in order to project an image on the area of the ceiling of the room in which there will be shown a virtual image of the moving sky, whether by day or by night, and which image, in the case of comprising three or more, is preferably a 3D virtual image or an image in the form of a hologram.

In another embodiment variant, the panel comprises a platform with one or more layers of LED lights joined with one another forming a network which, when connected to one or more pieces of control software, work in perfect harmony such that they form a clear moving natural image while some lights turn off and others turn on gradually.

Furthermore, in an embodiment option, the invention contemplates the introduction of a system for emitting odours through the air outlet nozzles, for which there is envisaged in the room the existence of a receptacle into which the user of the shower can introduce a container with an essence, for example, vanilla, menthol, etc., and with a device which suctions the essence and discharges it into the room through the nozzles.

It should be mentioned that, both in this option and in the preceding option, the panel of the invention is activated by means of a digital control unit which is preferably integrated in the wall of the room along with the water actuation unit which in turn activates and controls the pressure of the flow, the temperature, optionally the sound volume, the Bluetooth, and the odour actuation, among others mentioned herein, which are preferably incorporated in a digital control unit with different pushbuttons for each option, where it must be pointed out that, preferably, the activation of the device providing the virtual image of the sky on the ceiling of the room, either in its day or night format option, is independent of the activation of the exit of water, allowing or not allowing the combination of one thing with another according to user preference.

Preferably, the digital control unit in the form of a window installed in the wall of the room has a first secret button located on one side of the frame of the window which, when pressed, causes the lateral or vertical opening of a receptacle or inner area adapted as a support and connection for housing containers with essences which are threaded to an inner tube connected to the virtual panel, suctioning the perfume in a controlled manner and gradually discharging it through the air nozzles. The activation and deactivation of the emission of perfume into the room in the form of air and/or vapour is activated through a second secret button located on another side of the frame.

Optionally, when the room is a lift car, the inclusion of an emergency button is envisaged which, when pressed in case of emergency, in addition to activating the alarm warning in a remote emergency centre and sending the location of the emergency, also activates, at the same time, the device for emitting odours along with the gradually increasing discharge of fresh air through the nozzles of the panel, as well as the automatic playback of a video in which a person performs meditation and breathing exercises for several minutes in an attempt to calm the user in emergency and/or jam-up situations.

In turn, when the room in which the panel is installed is a shower and/or bathtub, a Bluetooth wireless communication module, which will allow the user to be able to play music in the room through a loudspeaker integrated in the virtual panel from a mobile device, is preferably integrated.

Likewise, when the room is a shower or bathtub, the incorporation of a lime filter in the water connection tube with the virtual panel is preferably envisaged, which will advantageously be linked to an alarm mechanism that will be activated periodically to warn the user of the need to change said filter from time to time, as foreseen.

### DESCRIPTION OF THE DRAWINGS

To complete the description, and for the purpose of helping to make the features of the invention more readily understandable, this description is accompanied by a set of drawings constituting an integral part of the same, which by way of illustration and not limitation represents the following:

FIG. 1 shows a schematic elevational cross-sectional depiction of the upper portion of a room consisting, for example, of a lift car, in which an exemplary embodiment of the virtual panel object of the invention has been installed, specifically an exemplary embodiment in which the panel only covers the ceiling of the car, where the parts and main elements thereof can be seen;

FIG. 2 shows a view similar to the foregoing which is a schematic cross-sectional depiction of the upper portion of a room consisting, for example, of a lift car, with the virtual panel of the invention installed therein, in this case showing an exemplary embodiment of the panel which covers the ceiling and a portion of one of the walls of the car;

FIG. 3 again shows a view similar to the foregoing which is a schematic sectional depiction of the upper portion of a room consisting, for example, of a lift car, with the virtual panel of the invention installed, in this case showing an exemplary embodiment of the panel which covers the ceiling and all the walls of the car;

FIG. 4 shows a front view of a wall of a room, specifically of a lift car, with the virtual panel of the invention installed, in which the mirror is shown integrated in the central panel which is activated or deactivated by double-clicking, allowing the panel function with the moving image to be activated and the mirror function to be deactivated, or vice versa;

FIG. 5 shows a schematic elevational and cross-sectional depiction of the upper portion of a room preferably comprising a shower or bathtub in which there has been installed an exemplary embodiment of the virtual panel of the invention, specifically an exemplary embodiment in which the panel comprises an upper screen, where the parts and main elements thereof can be seen;

FIG. 6 shows a schematic exploded perspective view of the upper screen of the panel of the invention and of the transparent platform with nozzles, according to the example shown in FIG. 4;

FIG. 7 shows a schematic elevational and cross-sectional depiction of the upper portion of a room preferably comprising a shower or bathtub in which another exemplary embodiment of the virtual panel of the invention has been installed, specifically an exemplary embodiment comprising a false ceiling with the network of lights integrated;

FIGS. 8-A and 8-B show schematic exploded perspective views of the false ceiling and the transparent platform with nozzles, according to the exemplary embodiment of the invention shown in FIG. 6, in FIG. 8-A and in another exemplary embodiment without a support disc in FIG. 8-B;

FIG. 9 shows a schematic elevational and cross-sectional depiction of the upper portion of a room preferably comprising a shower or bathtub in which there has been installed another exemplary embodiment of the virtual panel of the invention, specifically an exemplary embodiment comprising side projectors;

FIG. 10 shows a schematic perspective view of the upper portion of the room, according to the exemplary embodiment shown in FIG. 8; and

FIG. 11 shows a front view of an exemplary embodiment of the digital control unit in which, optionally, the control units of the virtual panel object of the invention are integrated, in an option which it is preferably used in a room comprising a shower or bathtub, together with the water flow and actuation control units of the shower or bathtub.

### PREFERRED EMBODIMENT OF THE INVENTION

In view of the mentioned figures and according to the numbering used, several non-limiting embodiments of the virtual panel for room of the invention can be observed therein, virtual panel which essentially comprises an electrical or electronic device (10, 12, 16) which, when activated, displays a moving image of a virtual representation of the sky or other natural or non-natural scenery, whether by day or by night; and furthermore including at least one outlet nozzle (6) for fluid which can be water or air, depending on the type of room (2).

Preferably, when the room (2) comprises a shower or bathtub, the panel contains one or more water outlet nozzles (6) and/or one or more air nozzles (6). In the case of having only air nozzles (6), the water for the shower or bathtub will come out through the conventional piping of the bathroom installation.

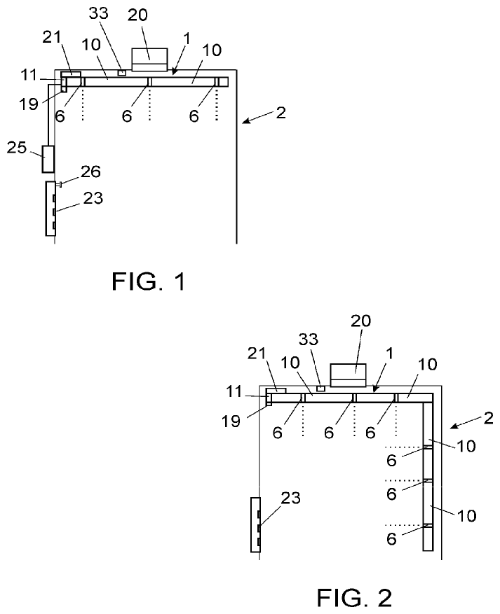

FIGS. 1-4 correspond to the preferred embodiment of the virtual panel with air of a room (2) consisting of a lift car.

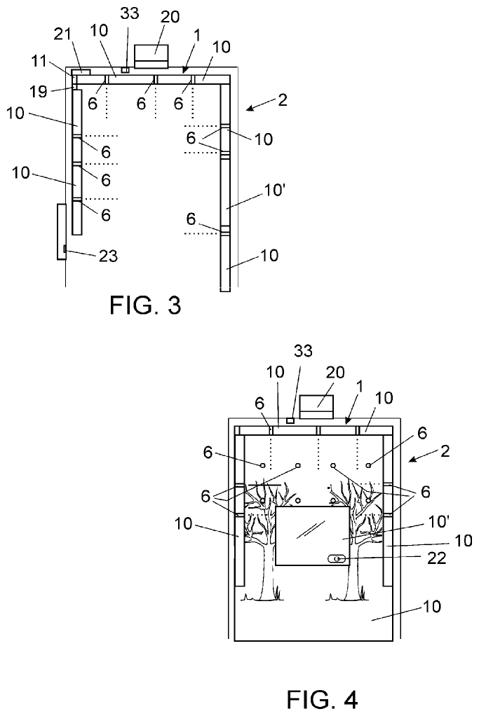

In a first embodiment option like the one shown in FIG. 1, the virtual panel (1) only covers the ceiling of the room (2) which preferably consists of a lift car; in another embodiment option like the one shown in FIG. 2, in addition to the ceiling of the room (2), the panel (1) also covers an upper portion or the entirety of at least one of the walls of the room (2), such that the image or images they display show continuity between both surfaces; and in a third embodiment option like the one shown in FIG. 3, the panel (1) covers the ceiling of the room (2) and the entirety of at least one or more of the walls of the room (2), such that the image which is displayed also shows continuity on all the surfaces it occupies. The buttons of the lift (23) are preferably located in a wall of the room surrounded by the panel (1). The air exiting through the nozzles originates from an air-conditioning machine (20) preferably located on the lift car.

In any case, said virtual images displayed by the panel (1) are preferably video images pre-recorded in a piece of software (21) contained in a playback and storage device (11) connected to at least one screen (10), which are optionally images produced with moving LED lights (13), or 3D images, or holograms produced by projectors (16).

Therefore, in the preferred embodiment like the one shown in FIGS. 1 to 4, the panel (1) comprises at least one image display screen (10) which is activated by means of a control unit (3), preferably integrated in the wall of the room (2).

Depending on the dimensions of the room (2) which consists preferably of a lift car, the panel (1) comprises a single screen (10) on which the moving virtual image is displayed, normally when it covers the ceiling of the room (2), and/or can be made up of a plurality of screens (10) placed next to one another covering the ceiling and, where appropriate, the wall or walls of the room (2), collectively showing the virtual image.

Preferably, in addition to images, the panel (1) also plays sound inside the room (2) through at least one loudspeaker (19) provided to that end, for example, connected to the video player (11), such that said sound is in synchronisation with the image.

Particularly when the room (2) is a lift car and the panel (1) completely or partially covers a wall thereof, said panel (1) preferably comprises a mirror screen (10′) consisting of a moving virtual touchscreen such that, when double-clicking said screen at a point (22) provided to that end, it stops displaying a portion of the moving image and transforms into a mirror, as shown in FIG. 4.

Optionally, when the room (2) is a lift car, it comprises a receptacle (25) with a device for emitting odours through the air nozzles (6). The user can introduce a container (28) with an essence, for example vanilla, menthol, etc. and, upon actuating the device (27), said device will suction said essence and meter same through the air nozzles (6) of the panel (1). Said receptacle has been schematically depicted in FIG. 1 and depicted in greater detail in FIG. 11 for the option of a room (2) such as a shower or bathtub.

Optionally, particularly when the room (2) is a lift car, this comprises an emergency button (26) which, in addition to activating an alarm in a remote centre and sending the location of the user, activates the device (27) to emit odours as well as to discharge fresh air which is gradually increased through the nozzles (6) of the panel, in an attempt to calm the user.

Preferably, the emergency button (26) in the room of the lift also activates the playback on the screen (10) of a video stored to that end in the storage player (11), in which a person performs breathing exercises in order to help calm the user.

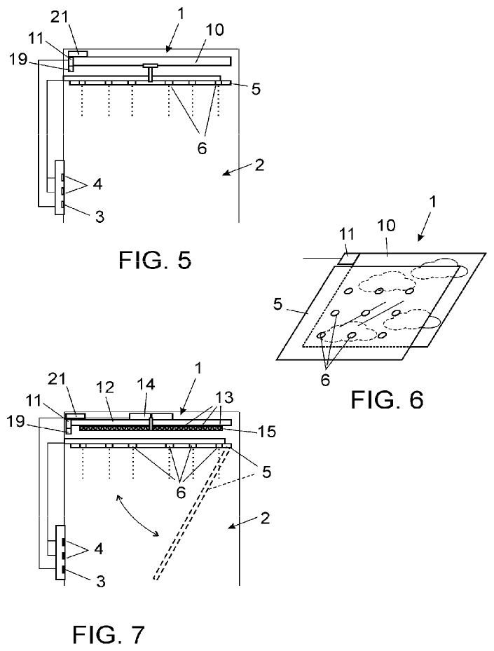

FIGS. 5-11 correspond to the preferred embodiment of a room comprising a shower or bathtub.

Preferably, when the room (2) comprises a shower or bathtub, it includes one or more water and/or air outlet nozzles (6).

Preferably, the nozzles (6) for fluid are arranged at the edges of the screen or screens (10) comprised in the panel (1), with the corresponding water conduit and/or air duct connection built into the wall or ceiling of the room (2).

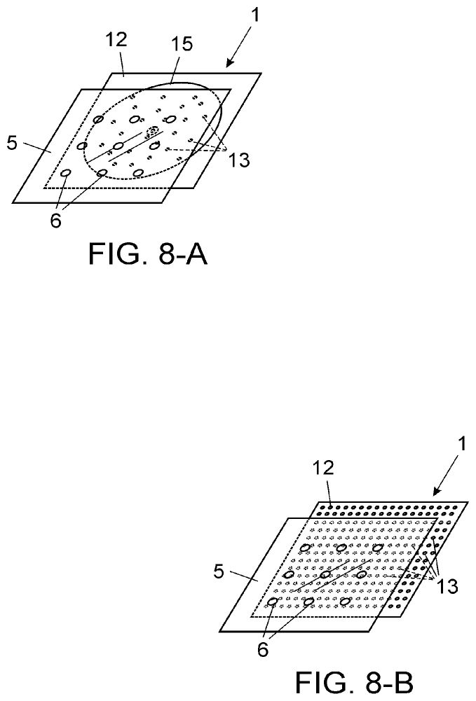

In reference to FIGS. 6 and 8, it can be seen how, in an embodiment for a room (2) comprising a shower or bathtub, the panel (1) contains an upper screen (10) suitable for displaying moving images of a sky with clouds and/or of a starry sky, pre-recorded in a piece of software (21) comprised in a playback and storage device (11) to which the screen (10) is connected which is activated by means of at least one control unit (3), preferably integrated in the wall of the room (2), as shown in FIGS. 5, 7, and 9, to which said player (11) is in turn connected.

Preferably, the screen (10) covers the entire upper portion of the room (2) comprising a shower or bathtub and is coupled built into the ceiling over a transparent platform (5) in which one or more water outlet nozzles (6) are integrated.

FIGS. 7 and 8 show how, in another embodiment variant also for a room (2) comprising a shower or bathtub, the panel (1) comprises a false ceiling (12) in which there are integrated a plurality of small lights (13), advantageously of LED type, which are connected to one another and to a motor (14) defining a rotational movement thereof, for example, about a support disc (15), emulating a moving starry sky.

In this variant, the false ceiling (12) with the network of lights (13) preferably covers the entire upper portion of the room (2) comprising a shower or bathtub and is coupled built into the ceiling over a transparent platform (5) in which one or more water outlet nozzles (6) are integrated.

Furthermore, said false ceiling (12) with the network of lights (13) is preferably activated by means of the control unit (3) which is preferably integrated in the wall of the room (2) comprising a shower or bathtub along with the water actuation units (4).

Optionally, the transparent platform (5), both in the variant with a screen (10) and in the variant with a false ceiling (12) with lights (13), can be a drop-down element to enable facilitating the cleaning of the screen (10) or of the false ceiling (12) with lights (13), as seen in FIG. 7, where said platform has been depicted in the usage position with continuous lines and in the dropped-down position with discontinuous lines.

Furthermore, preferably, when the room (2) is a shower or bathtub, the existence of a concealing glass is envisaged on the screen (10) or the false ceiling (12) with lights (13), which can be the glass forming the aforementioned platform (5) with the nozzles (6) or another consisting of an intermediate layer between said platform (5) and the screen (10) or the false ceiling (12) with lights, said glass being of the type which becomes opaque or translucent when an electric current is applied thereto, such that when the screen (10) or the false ceiling (12) with lights (13) is inactive, said glass becomes opaque and conceals the screen (10) or the false ceiling (12), being completely integrated in the ceiling of the room (2).

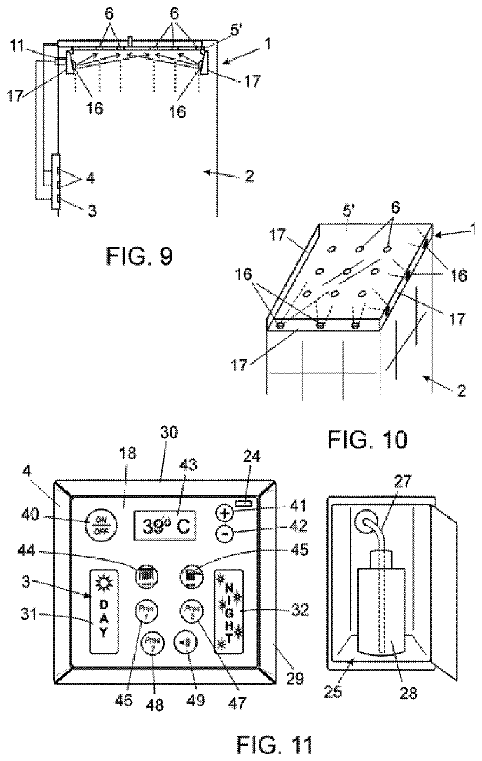

FIGS. 9 and 10 show how, in another embodiment variant also applicable for a room (2) comprising a shower or bathtub, the panel (1) of the invention contains a set of projectors (16) installed laterally in one or more plates (17) descending from the panel and preferably oriented at a 45° angle, when the panel (1) is activated by means of the control unit with a suitable inclination for projecting, on the surface of the upper area of the room (2) comprising a shower or bathtub, moving images, for example of a sky with clouds and/or a starry sky, whether by day or by night, pre-recorded in the playback and storage device (11) to which said projectors (16) are connected. Preferably, the panel (1) and the projectors (16) form an assembly to facilitate installation.

In this case, the image projected by the projectors (16) is depicted on the lower surface of a transparent or non-transparent platform (5′) in which one or more water and/or air outlet nozzles (6) are integrated.

Furthermore, also preferably, said projectors (16) are activated by means of a control unit (3) which is preferably integrated in the wall of a room (2) including a shower or bathtub along with the water actuation units (4).

Preferably, the control unit (3) regulating the actuation of the panel (1) is incorporated in the wall of the room (2) comprising a shower or bathtub, integrated in a small digital control unit (18) emulating a digital window that depicts the same moving image of the panel (1) in the background, both by day and by night.

Optionally, the virtual panel (1) is activated manually with the control unit (3) if the room (2) is a shower or bathtub and automatically by means of a movement or presence sensor if the room (2) is a lift.

Optionally, the panel (1) is connected to the lighting of the room (2) comprising a shower or bathtub such that, when the panel (1) is activated, the intensity of light of the entire room is automatically reduced by 50% or more.

FIG. 11 shows how, in a preferred embodiment for a room (2) comprising a shower or bathtub, the panel (1) of the invention, control is performed from a control unit set (3) integrated in a digital control unit (18) which is built into the wall of the room (2) comprising a shower or bathtub, preferably comprising at least one of the following buttons:
an on/off switch (40) for turning the virtual panel and the water flow on and off,
pushbuttons for increasing (41) and pushbuttons for reducing (42) the temperature shown on a digital screen (43),
pushbuttons for selecting water output, one for causing the output of water through a fixed point of the ceiling (44) and another from a manual securing point (45),
pushbuttons for selecting water pressure, preferably three, one low pressure pushbutton (46), one medium pressure pushbutton (47), and one high pressure pushbutton (48),
a button (49) for activating, increasing, and deactivating the sound of the panel (1), and
pushbuttons for selecting the image format of the virtual panel, one day format pushbutton (31) and one night format pushbutton (32),
a button (29) which, when pressed, causes the lateral or vertical opening of a receptacle (25) for housing containers (28) with essences,
a button (30) which activates and deactivates the emission of perfume into the room (2),
a button (4) for deactivating and separating the water flow from the operation of the moving virtual panel,
a button (24) which activates the Bluetooth wireless communication module.

Preferably, when the room (2) is a shower or bathtub, the panel (1) comprises a Bluetooth wireless communication module (24), for example, incorporated in the digital control unit (18), to enable playing music through the loudspeaker (19) from a mobile device.

Optionally, when the room (2) is a shower or bathtub, but also when the room is a lift car as set forth above, it comprises, for example, along with the digital control unit (18), as shown in FIG. 11, a receptacle (25) with a device (27) for suctioning and emitting odours through the air nozzles (6). The user can introduce a bottle (28) with an essence, for example vanilla, menthol, etc. and the device (27) will suction said essence and meter it through the air nozzles (6) provided in the room (2), along with the water nozzles (6).

Preferably, the digital control unit in the form of a window installed in the wall contains a first secret button (29) representing one side of the frame of the window which, when pressed, causes the lateral or vertical opening of a receptacle (25) for housing containers (28) with essences which incorporates a suction device (27) for emitting odours through the air nozzles (6), for example, an inner tube which suctions the perfume. The activation and deactivation of the emission of perfume in the room (2) in the form of air and/or vapour is activated through a second secret button (30) located on another side of the frame.

Preferably, when the digital control unit is in the form of a window installed in the wall, the digital control unit has a third secret button (4) for deactivating and separating the water flow from the operation of the virtual panel.

Preferably, when the room (2) is a shower or bathtub, the existence of a lime filter (not depicted) at the water outlet is envisaged, which is linked to an alarm mechanism, to periodically alert the user of the need to change said filter, preferably showing a warning in the digital control unit (18).

Optionally, the panel (1) comprises a microphone inhibiting device which can be activated so that the user can be assured that he or she cannot be heard by a third party.

The technical features described in an embodiment of a specific room can be used in any type of room.

Having sufficiently described the nature of the present invention, as well as the ways of implementing it, it is not considered necessary to expand its explanation for any person skilled in the art to understand its scope and the advantages which derive from it.

1. A virtual panel for ceilings and/or walls with water and/or air which can be installed as an accessory in an existing room (2) or during the installation process of said room, characterised in that it comprises an electrical or electronic device (10, 12, 16) which, when activated, displays a moving image of a virtual representation of the sky or other natural or non-natural scenery, whether by day or by night; and in that it further comprises at least one outlet nozzle (6) for fluid, which may be water and/or air, depending on the type of room (2).

2. The virtual panel for ceilings and/or walls with air according to claim 1, characterised in that it comprises one or more air outlet nozzles (6).

3. The virtual panel for ceilings and/or walls with air according to claim 1, characterised in that it only covers the ceiling of the room (2).

4. The virtual panel for ceilings and/or walls with air according to claim 1, characterised in that, in addition to a ceiling of the room (2), it also covers a portion of at least one of the walls of the room (2), such that the image which is displayed shows continuity between both surfaces.

5. The virtual panel for ceilings and/or walls with air according to claim 4, characterised in that it covers the ceiling of the room (2) and the entirety of at least one of the walls of the room (2), such that the image which is displayed shows continuity on all the surfaces it occupies.

6. The virtual panel for ceilings and/or walls with air according to claim 1, characterised in that it displays video images pre-recorded in a playback and storage device (11) connected to at least one screen (10).

7. The virtual panel for ceilings and/or walls with air according to claim 1, characterised in that it comprises at least one image display screen (10) which is activated by means of a movement sensor.

8. The virtual panel for ceilings and/or walls with air according to claim 1, characterised in that it comprises a single screen (10) on which the moving virtual image is displayed, or a plurality of screens (10) connected to one another covering the ceiling and, where appropriate, the wall or walls of the room (2), collectively showing the virtual image.

9. (canceled)

10. The virtual panel for ceilings and/or walls with air according to claim 1, characterised in that it comprises at least one of: at least one loudspeaker (19) which plays sound inside the room (2) in synchronisation with the image, a manual activation control unit (3), a mirror screen (10′) having of a touchscreen such that, when double-clicking said screen, it stops displaying the image and transforms into a mirror, a receptacle (25) for housing containers (28) with essences, which incorporates a suction device (27) for emitting odours through the air nozzles (6), or an emergency button (26) the activation of which, in addition to activating an alarm in an emergency centre and sending the location of the emergency, activates a device (27) for emitting odours and discharging air through the air nozzles (6).

11-14. (canceled)

15. The virtual panel for ceilings and/or walls with air according to claim 10, characterised in that the emergency button (26) is present and activation of the emergency button (26) also activates the playback of a pre-recorded video on the screen (10) as support wherein a person performs meditation and breathing exercises in order to help calm the user.

16. The virtual panel for ceilings and/or walls with water and/or air according to claim 1, characterised in that the room (2) is a shower or a bathtub and in that it comprises one or more water outlet nozzles (6).

17. The virtual panel for ceilings and/or walls with water and/or air according to claim 16, characterised in that the water nozzles (6) are arranged in a screen or screens (10) located in the ceiling comprising the panel (1), with the corresponding water conduit connection built into the wall or ceiling of the room (2).

18. The virtual panel for ceilings and/or walls with water and/or air according to claim 16, characterised in that a screen (10) is located in the ceiling and covers the entire upper portion of the room (2) and is incorporated in the ceiling on a transparent platform (5) in which one or more water outlet nozzles (6) are integrated.

19. The virtual panel for ceilings and/or walls with water and/or air according to claim 16, characterised in that it comprises a false ceiling (12) in which a network of small lights (13), which are connected to one another, are integrated, or a concealing glass or smart foil which becomes opaque or translucent when an electric current is applied thereto.

20. (canceled)

21. (canceled)

22. The virtual panel for ceilings and/or walls with water and/or air according to claim 18, characterised in that the transparent platform (5) in which the nozzles (6) are integrated is a drop-down element.

23. (canceled)

24. The virtual panel for ceilings and/or walls with water and/or air according to claim 16, characterised in that it comprises a set of projectors (16) integrated on the sides of the panel (1) in one or more plates (17) which are oriented at a 45° angle with respect to the panel (1) when the panel (1) is activated by means of the control unit, and project on the surface of the upper area of the room (2) moving images pre-recorded in the playback and storage device (11) to which said projectors (16) are connected.

25. The virtual panel for ceilings and/or walls with water and/or air according to claim 24, characterised in that the image they project is a 3D image or hologram or the image projected by the projectors (16) is shown on the lower surface of a transparent platform (5) or non-transparent platform (5′) in which one or more water outlet nozzles (6) are integrated.

26. (canceled)

27. The virtual panel for ceilings and/or walls with water and/or air according to claim 16, characterised in that it comprises a control unit (3) which regulates the actuation thereof, which is incorporated in the wall of the room (2), integrated in a digital control unit (18) emulating a window, or is connected to the lighting of the room (2), such that, when the panel (1) is activated, the intensity of light is reduced by 50% or more.

28. The virtual panel for ceilings and/or walls with water and/or air according to claim 27, characterised in that the digital control unit (3) comprises at least one of the following buttons;
an on/off switch (40) for turning the virtual panel and the water flow on and off,
pushbuttons for increasing (41) and pushbuttons for reducing (42) the temperature, shown on a digital screen (43),
pushbuttons for selecting water output, one for causing the output of water through a fixed point of the ceiling (44) and another from a manual securing point (45),
pushbuttons for selecting water pressure, preferably three, one low pressure pushbutton (46), one medium pressure pushbutton (47), and one high pressure pushbutton (48),
a button (49) for activating, increasing, and deactivating the sound of the panel (1), and
pushbuttons for selecting the image format of the virtual panel, one day format pushbutton (31) and one night format pushbutton (32).
a button (29) which, when pressed, causes the lateral or vertical opening of a receptacle (25) for housing containers (28) with essences,
a button (30) which activates and deactivates the emission of perfume into the room (2),
a button (4) for deactivating and separating the water flow from the operation of the moving virtual impanel, or
a button (24) which activates the Bluetooth wireless communication module.

29. (canceled)

30. The virtual panel for ceilings and/or walls with water and/or air according to claim 16, characterised in that it comprises at least one of: a microphone inhibiting device which can be activated so that the user can be assured that he or she cannot be heard by a third party, a Bluetooth wireless communication module (24) to play music through a loudspeaker (19) from a mobile device, a lime filter at the water outlet, linked to an alarm mechanism, to periodically alert the user of the need to change said filter, or a receptacle (25) for housing containers (28) with essences, which incorporates a suction device (27) for emitting odours through air nozzles (6) provided along with the water nozzles (6) in the form of air and/or vapour.

31-33. (canceled)
